(19) United States
(12) Patent Application Publication
(10) Pub. No.: US 2017/0231580 A1
(43) Pub. Date: Aug. 17, 2017

(54) Methods and Systems for Electrophysiology Mapping Using Medical Images

(71) Applicant: St. Jude Medical, Cardiology Division, Inc., St. Paul, MN (US)

(72) Inventor: Jatin Surendra Relan, Bordeaux (FR)

(21) Appl. No.: 15/431,947

(22) Filed: Feb. 14, 2017

Related U.S. Application Data
(60) Provisional application No. 62/295,852, filed on Feb. 16, 2016.

(51) Int. Cl. A61B 5/00; G06T 7/37; A61B 6/03; A61B 5/06; A61B 5/042; A61B 5/055

(52) U.S. Cl. CPC A61B 5/743; A61B 5/0422; A61B 5/055; A61B 6/032; A61B 5/066; G06T 7/37; A61B 2576/023; G06T 2207/10088; G06T 2207/10081; G06T 2207/30048; G06T 2207/20221; G06T 2207/10076

(57) ABSTRACT

A method of displaying electrophysiology information includes obtaining a three-dimensional medical image of an anatomical region, registering a localization system to the model; localizing an electrophysiology catheter within the anatomical region; displaying a representation of the localization of the electrophysiology catheter on the model; and displaying image slices of the model. The image slices are selected based upon the localization of the electrophysiology catheter. For example, the image slices can pass through a user-selected localization element carried by the electrophysiology catheter. Rigid and/or non-rigid transforms can be used to register the localization system to the model. Electrophysiology data collected by the catheter can be displayed on the model and/or the image slices thereof. The three-dimensional medical image and/or the electrophysiology data can also be time-varying. In embodiments, scalar maps can also be displayed on the model.

### CROSS-REFERENCE TO RELATED APPLICATIONS

This application claims the benefit of U.S. provisional application No. 62/295,852, filed 16 Feb. 2016, now pending, which is hereby incorporated by reference as though fully set forth herein.

### BACKGROUND

The instant disclosure relates to electrophysiological mapping, such as may be performed in cardiac diagnostic and therapeutic procedures. In particular, the instant disclosure relates to methods, apparatuses, and systems for displaying electrophysiology maps on medical images, such as computerized tomography and magnetic resonance images.

Cardiac electrical disorders are a major cause of human morbidity and mortality worldwide. Those of ordinary skill in the art will be familiar with the practice of diagnosing cardiac electrical disorders through electrophysiology mapping using catheter-borne contact and/or non-contact electrodes. It would be desirable, however, to combine information from a cardiac electrophysiology study with information from a high resolution medical image, such as a computerized tomography or magnetic resonance image.

### BRIEF SUMMARY

Disclosed herein is a method of displaying electrophysiology information, including: registering a localization system to a three-dimensional medical image of an anatomical region; localizing an electrophysiology catheter within the anatomical region using the localization system; displaying a representation of the localization of the electrophysiology catheter on the three-dimensional medical image of the anatomical region; and displaying one or more image slices of the three-dimensional medical image of the anatomical region, the one or more image slices being selected based upon the localization of the electrophysiology catheter. The three-dimensional medical image of the anatomical region can be a magnetic resonance image, a computerized tomography image, or another medical image. The three-dimensional medical image of the anatomical region can also be a time-varying image (that is, an image that depicts the changing geometry of the anatomical region over time, such as the beating of a heart).

According to aspects of the disclosure, a non-rigid transformation is used to register the localization system to the three-dimensional medical image of the anatomical region. Non-rigid transformations include, without limitation, thin plate splines transforms, radial basis function transforms, and mean value coordinate transforms. In other aspects of the disclosure, a rigid transformation is used to register the localization system to the three-dimensional medical image of the anatomical region.

It is contemplated that the one or more image slices can be taken through a location of a preselected localization element carried by the electrophysiology catheter.

Electrophysiology data measured by the electrophysiology catheter can be displayed on the three-dimensional medical image of the anatomical region and/or the one or more image slices of the three-dimensional medical image of the anatomical region. For example, a plurality of glyphs can be used to display the electrophysiology data on the three-dimensional medical image and/or the image slices thereof.

According to additional aspects of the disclosure, one or more scalar maps can be displayed on the three-dimensional medical image of the anatomical region.

Also disclosed herein is a method of displaying electrophysiology information, including: registering a localization system to a three-dimensional medical image of an anatomical region; localizing an electrophysiology catheter within the anatomical region using the localization system, the electrophysiology catheter including at least one localization element; displaying one or more image slices of the three-dimensional medical image of the anatomical region, wherein the one or more image slices pass through a localization of the at least one localization element; and displaying electrophysiology information measured by the electrophysiology catheter on at least one of the three-dimensional medical image of the anatomical region and the one or more image slices of the three-dimensional medical image of the anatomical region. The method can also include displaying a representation of the localization of the electrophysiology catheter on at least one of the three-dimensional medical image of the anatomical region and the one or more image slices of the three-dimensional medical image of the anatomical region.

According to aspects of the disclosure, the localization system can be registered to the three-dimensional medical image of the anatomical region using a non-rigid transformation, such as a thin plate splines transform, a radial basis function transform, and a mean value coordinate transform.

Exemplary three-dimensional medical images include, without limitation, computerized tomography images, optical coherence tomography images, magnetic resonance images, ultrasound images, x-ray images, fluoroscopic images, image templates, localization system generated images, segmented models of any of the foregoing, and any combinations thereof.

According to another embodiment of the disclosure, an electrophysiology system includes: a localization system configured to localize an electrophysiology catheter; a registration processor configured to register the localization system to a three-dimensional medical image of an anatomical region; and a mapping processor configured to display: a representation of a localization of the electrophysiology catheter on the three-dimensional medical image of the anatomical region; and one or more image slices of the three-dimensional medical image of the anatomical region, the one or more image slices being selected based upon the localization of the electrophysiology catheter. The mapping processor can further be configured to display electrophysiology data measured by the electrophysiology catheter on at least one of the three-dimensional medical image of the anatomical region and the one or more image slices of the three-dimensional medical image of the anatomical region. In embodiments of the disclosure, the representation of the localization of the electrophysiology catheter and the one or more image slices of the three-dimensional medical image of the anatomical region are time-varying.

The foregoing and other aspects, features, details, utilities, and advantages of the present invention will be apparent from reading the following description and claims, and from reviewing the accompanying drawings.

### BRIEF DESCRIPTION OF THE DRAWINGS

FIG. 1 is a schematic of an exemplary electroanatomical mapping system, such as may be used in an electrophysiology study.

FIG. 2 depicts an exemplary catheter that can be used in an electrophysiology study.

FIG. 3 is a flowchart of representative steps that can be followed to display information collected during an electrophysiology study on a medical image, such as a three-dimensional image of an anatomical region.

FIGS. 4A and 4B depict the registration of a localization system to a three-dimensional medical image.

FIG. 5 illustrates the presentation of electrophysiology study information, including catheter location, on a three-dimensional medical image and several slices thereof.

FIG. 6 depicts the presentation of electrophysiology data on a three-dimensional medical image and several slices thereof.

FIG. 7 depicts a scalar map displayed on a three-dimensional medical image.

FIG. 8 depicts time varying or dynamic electrophysiology study information presented on a three-dimensional medical image and several slices thereof.

### DETAILED DESCRIPTION

The present disclosure provides methods, apparatuses, and systems for the creation of electrophysiology maps. For purposes of illustration, several exemplary embodiments will be described in detail herein in the context of cardiac electrophysiology. It is contemplated, however, that the systems, methods, and apparatuses described herein can be utilized in other contexts.

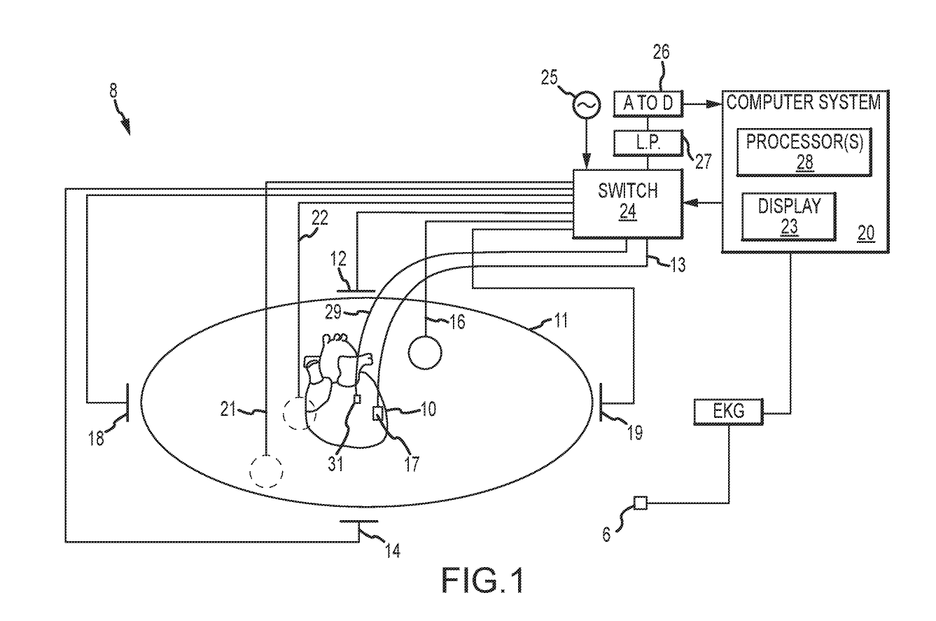

FIG. 1 shows a schematic diagram of an electrophysiology system 8 for conducting cardiac electrophysiology studies by navigating a cardiac catheter and measuring electrical activity occurring in a heart 10 of a patient 11 and three-dimensionally mapping the electrical activity and/or information related to or representative of the electrical activity so measured. System 8 can be used, for example, to create an anatomical model of the patient's heart 10 using one or more electrodes. System 8 can also be used to measure electrophysiology data, including, but not limited to, local activation time (“LAT”), at a plurality of points along a cardiac surface and store the measured data in association with location information for each measurement point at which the electrophysiology data was measured, for example to create a diagnostic data map of the patient's heart 10.

As one of ordinary skill in the art will recognize, and as will be further described below, system 8 can determine the location, and in some aspects the orientation, of objects, typically within a three-dimensional space, and express those locations as position information determined relative to at least one reference.

For simplicity of illustration, the patient 11 is depicted schematically as an oval. In the embodiment shown in FIG. 1, three sets of surface electrodes (e.g., patch electrodes) are shown applied to a surface of the patient 11, defining three generally orthogonal axes, referred to herein as an x-axis, a y-axis, and a z-axis. In other embodiments the electrodes could be positioned in other arrangements, for example multiple electrodes on a particular body surface. As a further alternative, the electrodes do not need to be on the body surface, but could be positioned internally to the body or on an external frame.

In FIG. 1, the x-axis surface electrodes 12, 14 are applied to the patient along a first axis, such as on the lateral sides of the thorax region of the patient (e.g., applied to the patient's skin underneath each arm) and may be referred to as the Left and Right electrodes. The y-axis electrodes 18, 19 are applied to the patient along a second axis generally orthogonal to the x-axis, such as along the inner thigh and neck regions of the patient, and may be referred to as the Left Leg and Neck electrodes. The z-axis electrodes 16, 22 are applied along a third axis generally orthogonal to both the x-axis and the y-axis, such as along the sternum and spine of the patient in the thorax region, and may be referred to as the Chest and Back electrodes. The heart 10 lies between these pairs of surface electrodes 12/14, 18/19, and 16/22.

An additional surface reference electrode (e.g., a “belly patch”) 21 provides a reference and/or ground electrode for the system 8. The belly patch electrode 21 may be an alternative to a fixed intra-cardiac electrode 31, described in further detail below. It should also be appreciated that, in addition, the patient 11 may have most or all of the conventional electrocardiogram (“ECG” or “EKG”) system leads in place. In certain embodiments, for example, a standard set of 12 ECG leads may be utilized for sensing electrocardiograms on the patient's heart 10. This ECG information is available to the system 8 (e.g., it can be provided as input to computer system 20). Insofar as ECG leads are well understood, and for the sake of clarity in the figures, only one lead 6 and its connection to computer system 20 is illustrated in FIG. 1.

A representative catheter 13 having at least one electrode 17 (e.g., a distal electrode) is also depicted in schematic fashion in FIG. 1. This representative catheter electrode 17 can be referred to as a “measurement electrode” or a “roving electrode.” Typically, multiple electrodes on catheter 13, or on multiple such catheters, will be used. In one embodiment, for example, system 8 may utilize sixty-four electrodes on twelve catheters disposed within the heart and/or vasculature of the patient.

In other embodiments, system 8 may utilize a single catheter that includes multiple (e.g., eight) splines, each of which in turn includes multiple (e.g., eight) electrodes. Of course, these embodiments are merely exemplary, and any number of electrodes and catheters may be used. Indeed, in some embodiments, a high density mapping catheter, such as the EnSite™ Array™ non-contact mapping catheter of St. Jude Medical, Inc., can be utilized.

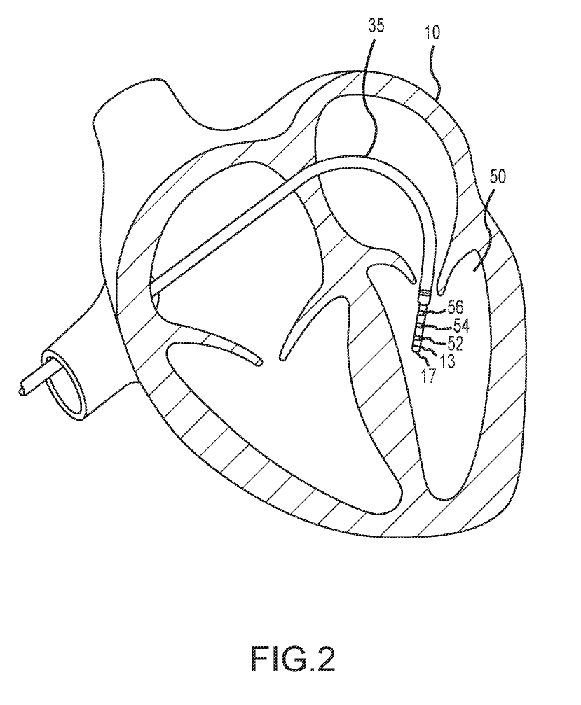

Likewise, it should be understood that catheter 13 (or multiple such catheters) are typically introduced into the heart and/or vasculature of the patient via one or more introducers and using familiar procedures. For purposes of this disclosure, a segment of an exemplary multi-electrode catheter 13 is shown in FIG. 2. In FIG. 2, catheter 13 extends into the left ventricle 50 of the patient's heart 10 through a transseptal sheath 35. The use of a transseptal approach to the left ventricle is well known and will be familiar to those of ordinary skill in the art, and need not be further described herein. Of course, catheter 13 can also be introduced into the heart 10 in any other suitable manner.

Catheter 13 includes electrode 17 on its distal tip, as well as a plurality of additional measurement electrodes 52, 54, 56 spaced along its length in the illustrated embodiment. Typically, the spacing between adjacent electrodes will be known, though it should be understood that the electrodes may not be evenly spaced along catheter 13 or of equal size to each other. Since each of these electrodes 17, 52, 54, 56 lies within the patient, location data may be collected simultaneously for each of the electrodes by system 8.

Similarly, each of electrodes 17, 52, 54, and 56 can be used to gather electrophysiological data from the cardiac surface. The ordinarily skilled artisan will be familiar with various modalities for the acquisition and processing of electrophysiology data points (including, for example, both contact and non-contact electrophysiological mapping), such that further discussion thereof is not necessary to the understanding of the conduction velocity mapping techniques disclosed herein. Likewise, various techniques familiar in the art can be used to generate a graphical representation from the plurality of electrophysiology data points. Insofar as the ordinarily skilled artisan will appreciate how to create electrophysiology maps from electrophysiology data points, the aspects thereof will only be described herein to the extent necessary to understand the maps disclosed herein.

Returning now to FIG. 1, in some embodiments, a fixed reference electrode 31 (e.g., attached to a wall of the heart 10) is shown on a second catheter 29. For calibration purposes, this electrode 31 may be stationary (e.g., attached to or near the wall of the heart) or disposed in a fixed spatial relationship with the roving electrodes (e.g., electrodes 17, 52, 54, 56), and thus may be referred to as a “navigational reference” or “local reference.” The fixed reference electrode 31 may be used in addition or alternatively to the surface reference electrode 21 described above. In many instances, a coronary sinus electrode or other fixed electrode in the heart 10 can be used as a reference for measuring voltages and displacements; that is, as described below, fixed reference electrode 31 may define the origin of a coordinate system.

Each surface electrode is coupled to a multiplex switch 24, and the pairs of surface electrodes are selected by software running on a computer 20, which couples the surface electrodes to a signal generator 25. Alternately, switch 24 may be eliminated and multiple (e.g., three) instances of signal generator 25 may be provided, one for each measurement axis (that is, each surface electrode pairing).

The computer 20, for example, may comprise a conventional general-purpose computer, a special-purpose computer, a distributed computer, or any other type of computer. The computer 20 may comprise one or more processors 28, such as a single central processing unit (CPU), or a plurality of processing units, commonly referred to as a parallel processing environment, which may execute instructions to practice the various aspects disclosed herein.

Generally, three nominally orthogonal electric fields are generated by a series of driven and sensed electric dipoles (e.g., surface electrode pairs 12/14, 18/19, and 16/22) in order to realize catheter navigation in a biological conductor. Alternatively, these orthogonal fields can be decomposed and any pairs of surface electrodes can be driven as dipoles to provide effective electrode triangulation. Likewise, the electrodes 12, 14, 18, 19, 16, and 22 (or any other number of electrodes) could be positioned in any other effective arrangement for driving a current to or sensing a current from an electrode in the heart. For example, multiple electrodes could be placed on the back, sides, and/or belly of patient 11. For any desired axis, the potentials measured across the roving electrodes resulting from a predetermined set of drive (source-sink) configurations may be combined algebraically to yield the same effective potential as would be obtained by simply driving a uniform current along the orthogonal axes.

Thus, any two of the surface electrodes 12, 14, 16, 18, 19, 22 may be selected as a dipole source and drain with respect to a ground reference, such as belly patch 21, while the unexcited electrodes measure voltage with respect to the ground reference. The roving electrodes 17, 52, 54, 56 placed in the heart 10 are exposed to the field from a current pulse and are measured with respect to ground, such as belly patch 21. In practice the catheters within the heart 10 may contain more or fewer electrodes than the four shown, and each electrode potential may be measured. As previously noted, at least one electrode may be fixed to the interior surface of the heart to form a fixed reference electrode 31, which is also measured with respect to ground, such as belly patch 21, and which may be defined as the origin of the coordinate system relative to which localization system 8 measures positions. Data sets from each of the surface electrodes, the internal electrodes, and the virtual electrodes may all be used to determine the location of the roving electrodes 17, 52, 54, 56 within heart 10.

The measured voltages may be used by system 8 to determine the location in three-dimensional space of the electrodes inside the heart, such as roving electrodes 17, 52, 54, 56, relative to a reference location, such as reference electrode 31. That is, the voltages measured at reference electrode 31 may be used to define the origin of a coordinate system, while the voltages measured at roving electrodes 17, 52, 54, 56 may be used to express the location of roving electrodes 17, 52, 54, 56 relative to the origin. In some embodiments, the coordinate system is a three-dimensional (x, y, z) Cartesian coordinate system, although other coordinate systems, such as polar, spherical, and cylindrical coordinate systems, are contemplated.

As should be clear from the foregoing discussion, the data used to determine the location of the electrode(s) within the heart is measured while the surface electrode pairs impress an electric field on the heart. The electrode data may also be used to create a respiration compensation value used to improve the raw location data for the electrode locations as described in U.S. Pat. No. 7,263,397, which is hereby incorporated herein by reference in its entirety. The electrode data may also be used to compensate for changes in the impedance of the body of the patient as described, for example, in U.S. Pat. No. 7,885,707, which is also incorporated herein by reference in its entirety.

In one representative embodiment, the system 8 first selects a set of surface electrodes and then drives them with current pulses. While the current pulses are being delivered, electrical activity, such as the voltages measured with at least one of the remaining surface electrodes and in vivo electrodes, is measured and stored. Compensation for artifacts, such as respiration and/or impedance shifting, may be performed as indicated above.

In some embodiments, system 8 is the EnSite™ Velocity™ cardiac mapping and visualization system of St. Jude Medical, Inc., which generates electrical fields as described above, or another localization system that relies upon electrical fields. Other localization systems, however, may be used in connection with the present teachings, including for example, systems that utilize magnetic fields instead of or in addition to electrical fields for localization. Examples of such systems include, without limitation, the CARTO navigation and location system of Biosense Webster, Inc., the AURORA® system of Northern Digital Inc., Sterotaxis' NIOBE® Magnetic Navigation System, as well as MediGuide™ Technology and the EnSite™ Precision™ system, both from St. Jude Medical, Inc.

The localization and mapping systems described in the following patents (all of which are hereby incorporated by reference in their entireties) can also be used with the present invention: U.S. Pat. Nos. 6,990,370; 6,978,168; 6,947,785; 6,939,309; 6,728,562; 6,640,119; 5,983,126; and 5,697,377.

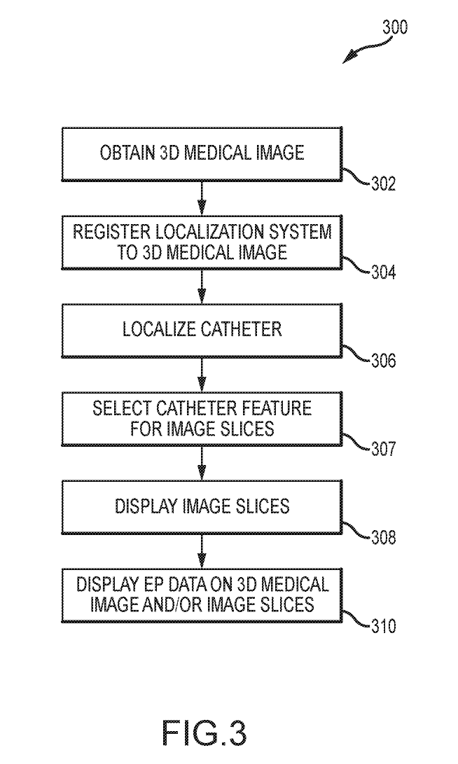

One basic methodology of displaying electrophysiology information on a medical image will be explained with reference to the flowchart 300 of representative steps presented as FIG. 3. In some embodiments, for example, the flowchart may represent several exemplary steps that can be carried out by the computer 20 of FIG. 1 (e.g., by one or more processors 28 executing one or more specialized modules, such as a registration processor executing a registration module or a mapping processor executing a mapping module as further described below) to display electrophysiology information on a medical image as described herein. It should be understood that the representative steps described below can be either hardware- or software-implemented. For the sake of explanation, the term “signal processor” is used herein to describe both hardware- and software-based implementations of the teachings herein. Likewise, it should be understood that the teachings herein can be executed on a single CPU, which may have one or more threads, or distributed across multiple CPUs, each of which may have one or more threads, in a parallel processing environment.

A three-dimensional medical image of an anatomical region (e.g., a portion of a patient's heart) is obtained in block 302. Various sources for the three-dimensional medical image are contemplated including, without limitation, magnetic resonance images, optical coherence tomography images, computerized tomography images, ultrasound images, x-ray images, fluoroscopic images, image templates, localization system generated images, segmented models of any of the foregoing, and combinations of any of the foregoing. Insofar as these imaging modalities will be familiar to the person of ordinary skill in the art, they are not explained in detail herein.

In block 304, a localization system (e.g., system 8) is registered to the three-dimensional medical image obtained in block 302. Various registration approaches, including both rigid and non-rigid transforms, are contemplated. According to aspects of the disclosure, the registration uses a thin plate splines transform, a radial basis function transform, or a mean value coordinate transform. U.S. application Ser. No. 11/715,923, which is hereby incorporated by reference as though fully set forth herein, describes the use of the foregoing non-rigid transforms to register localization systems to medical images in detail.

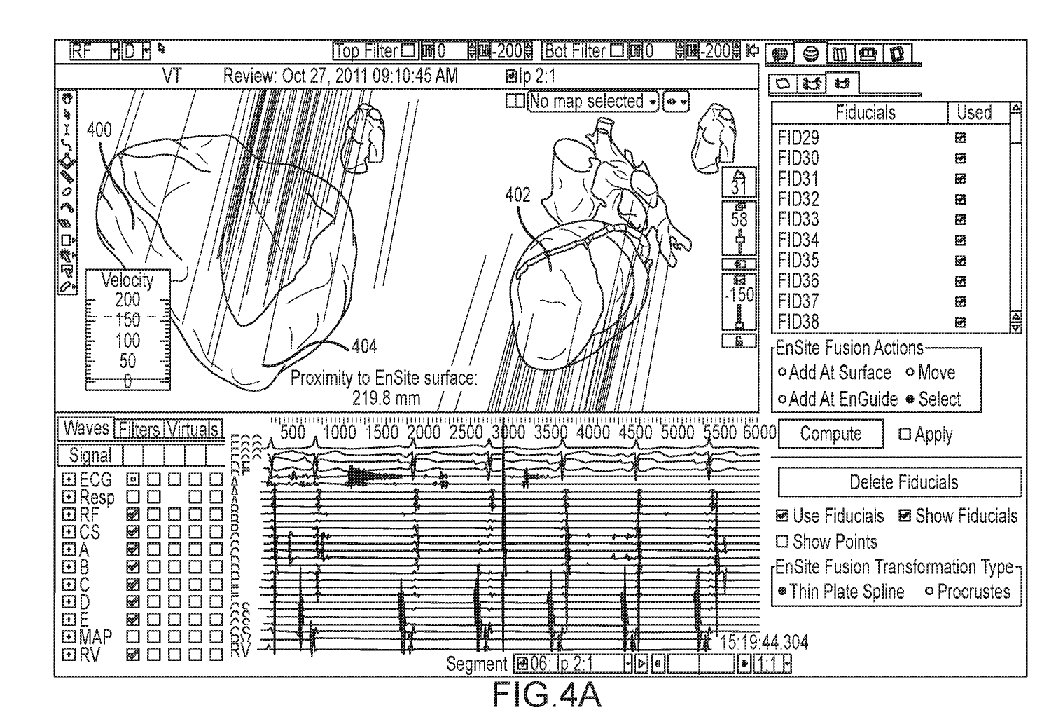

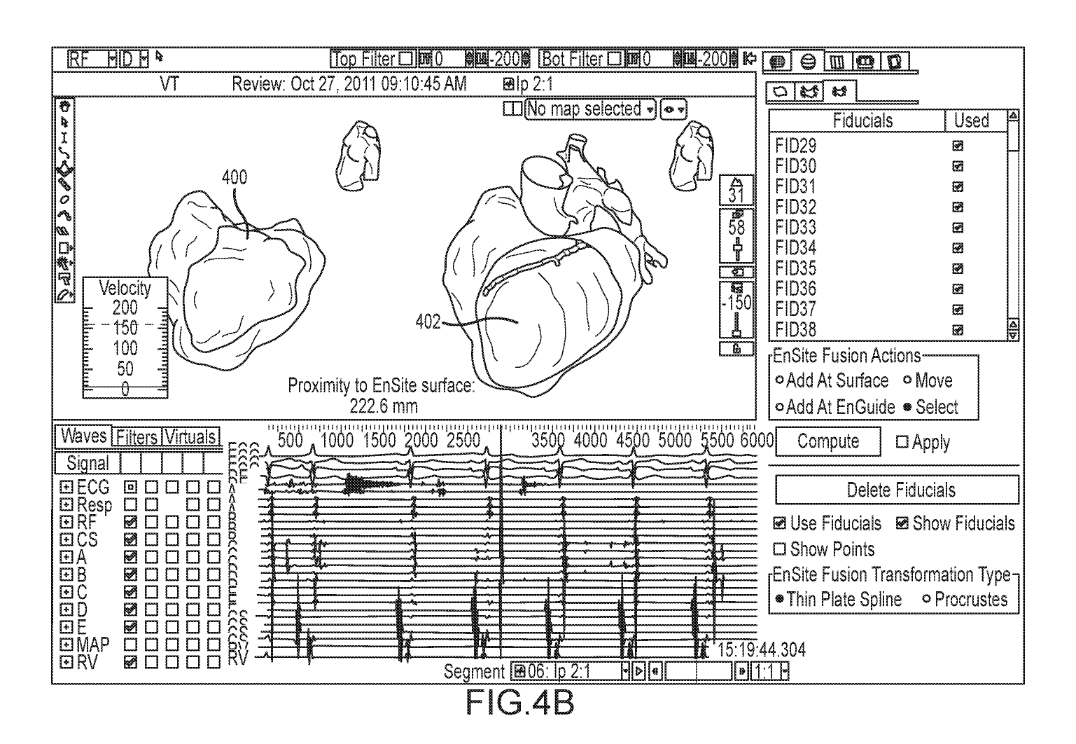

FIG. 4A depicts a model of the heart 400, generated by system 8, alongside a three-dimensional medical image of the heart 402, such as a magnetic resonance or computerized tomography image. FIG. 4A also shows the mapping of a plurality of fiducial points 404 on model 400 to three-dimensional medical image 402. FIG. 4B shows the results of the registration. Those of ordinary skill in the art will appreciate that these images may be output to display 23 as part of a graphical user interface (“GUI”), which may contain one or more windows; exemplary embodiments will be described below.

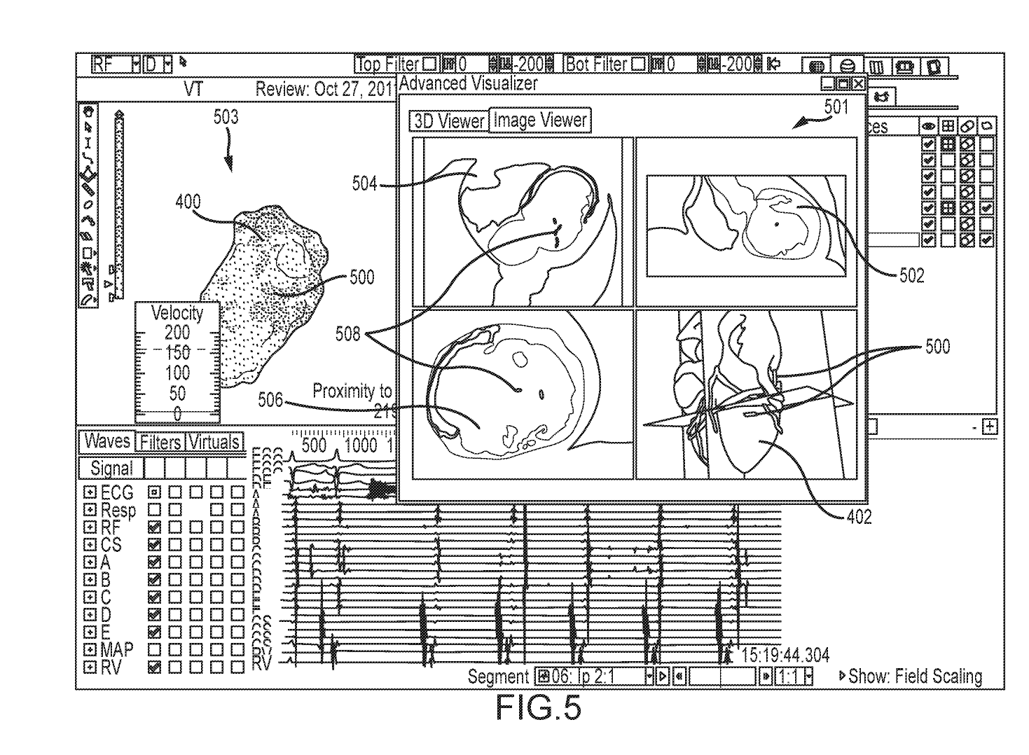

Returning now to flowchart 300 in FIG. 3, in block 306, an electrophysiology catheter, such as catheter 13, is localized using system 8. The registration computed in block 304 can be used to display (e.g., on display 23) a graphical representation(s) 500 of catheter(s) 13 in the registered three-dimensional medical image 402 as shown in the lower right-hand quadrant of the overlying window 501 shown in FIG. 5. A graphical representation(s) 500 of catheter(s) 13 can also be shown in the model of the heart 400 generated by system 8, such as depicted towards the left-hand side of the underlying window 503 shown in FIG. 5.

Overlying window 501 in FIG. 5 can be further sub-divided (e.g., into quadrants) in order to depict one or more image slices 502 (coronal plane), 504 (axial plane), 506 (sagittal plane) of the three-dimensional medical image of the anatomical region 402 (block 308) and/or registered three-dimensional medical image 402. Image slices 502, 504, and 506 can also include graphical representations 508 of catheter(s) 13.

Image slices 502, 504, and 506 can be selected based upon the localization of catheter 13. More particularly, according to aspects of the disclosure, image slices 502, 504, and 506 can be selected to pass through a preselected feature on catheter 13, such as one or more of electrodes 17, 52, 54, 56. This feature can be user-selected in block 307. Slices 502, 504, and 506 can be taken automatically (e.g., as part of a visualization or other module executing on one or more processors 28) or manually (e.g., by allowing the user to “point-and-click” within the GUI to identify a point through which slices 502, 504, and 506 should be taken).

Those of ordinary skill in the art will appreciate that a segmented model is made up of a number of discrete image slices. It is possible, therefore, that there may be no image slice that passes directly through the location of the preselected localization element or other catheter feature. For purposes of the instant disclosure, therefore, an image slice “passes through” the location of a preselected feature if it is the closest image slice to the location of the preselected feature, even if the feature does not fall squarely within the plane of the image slice.

An image slice can also be interpolated from the image volume, using a user-defined plane along the field of view of a catheter 13. This can be advantageous, for example, to the visualization of scar heterogeneity with the myocardium.

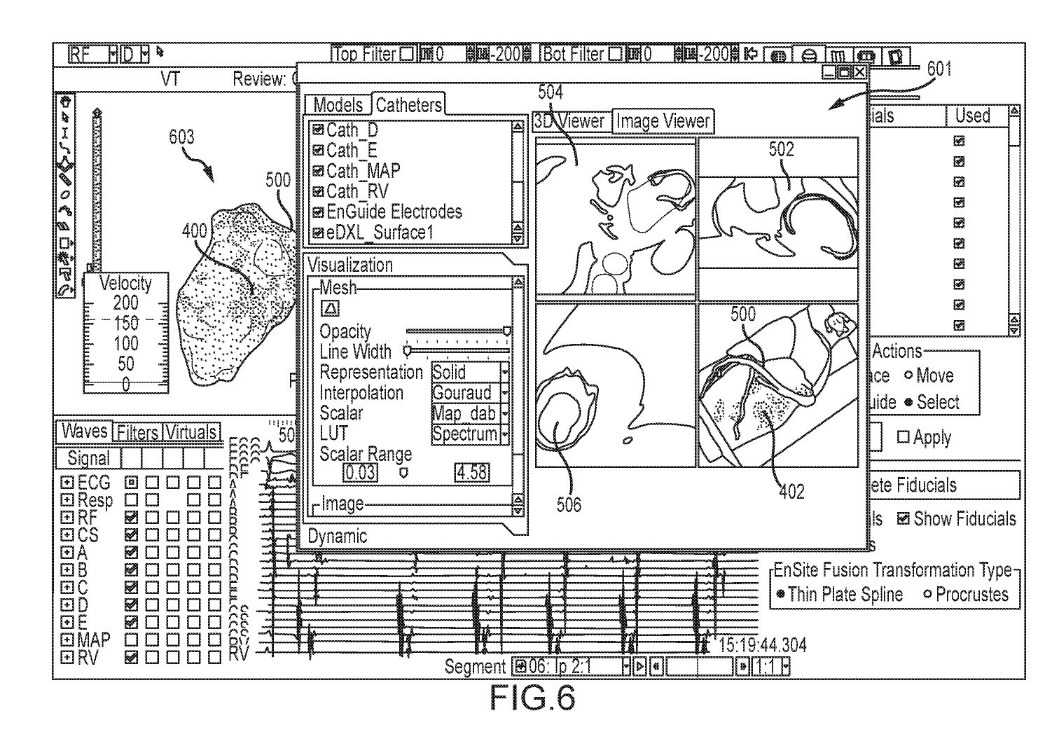

In block 310, electrophysiology data can be displayed on three-dimensional medical image 402 and/or image slices 502, 504, 506. For example, according to some embodiments of the disclosure, the electrophysiology data is displayed using a plurality of glyphs, such as disclosed in U.S. application Ser. No. 14/611,597, which is hereby incorporated by reference as though fully set forth herein. FIG. 6 depicts the inclusion of electrophysiology data on three-dimensional medical image 402 in a multiple window view similar to that shown in FIG. 5 (e.g., an overlying window 601 sub-divided into quadrants to depict several image slices 502, 504, 506 and the registered three-dimensional medical image 402, and a primary window 603 that shows the model of the heart 400 as generated by system 8, which can also include electrophysiology data). Insofar as the ordinarily skilled artisan will be familiar with various modalities for the acquisition and processing of electrophysiology data (including, for example, both contact and non-contact electrophysiological mapping), as well as with various techniques that can be used to generate a graphical representation of the acquired electrophysiology data, these aspects will not be further described herein.

Scalar maps can also be displayed on three-dimensional medical image 402. For example, a contrast material-enhanced cardiac-gated multi-detector CT image, which can depict cardiac anatomy with high spatial resolution and thus depict cardiac wall thinning, can be displayed on the three-dimensional medical image 402. As another example, a delayed contrast-enhanced MR image, which can be used to assess focal myocardial fibrosis, the substrate involved in most cardiac arrhythmias, can be displayed on the three-dimensional medical image 402. That is, a wall thinning scalar map and/or a myocardial fibrosis scalar map can be displayed on the three-dimensional medical image 402.

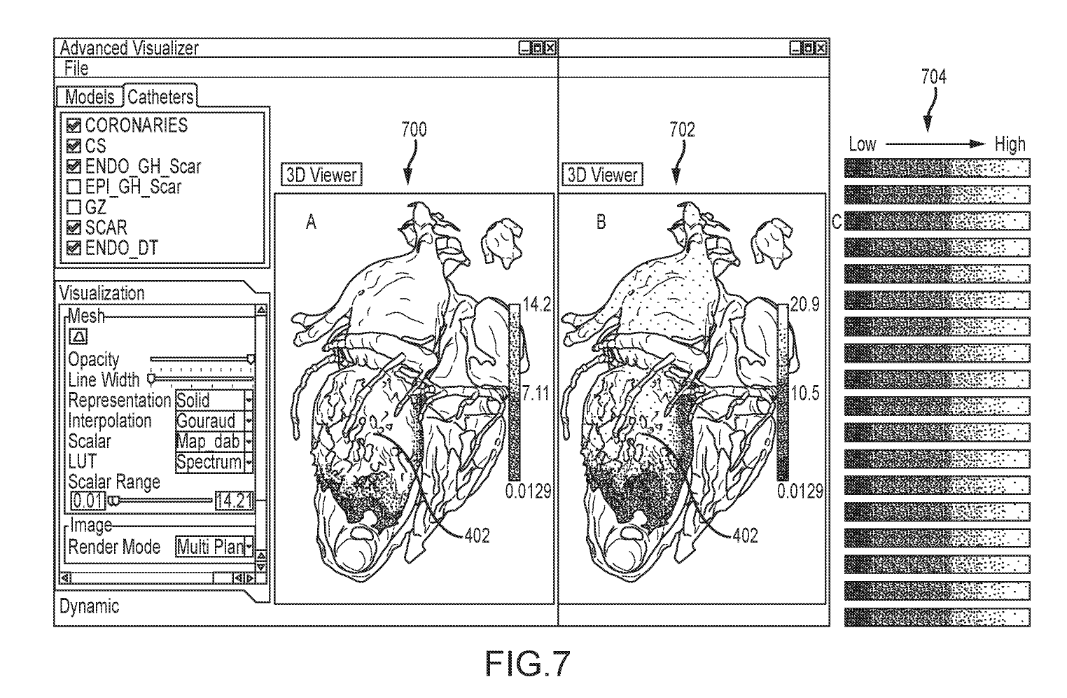

FIG. 7 depicts exemplary scalar maps 700 and 702. Panel 704 in FIG. 7 shows various color look-up tables that can be used to draw scalar maps 700 and 702 onto the three-dimensional medical image 402.

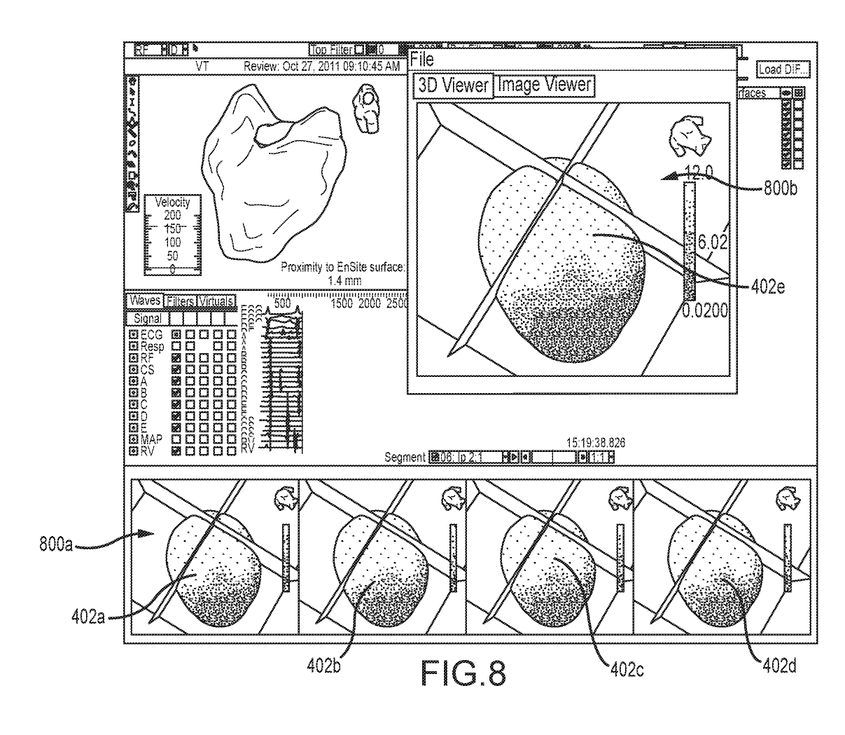

It is also contemplated that the geometry of the three-dimensional medical image 402 and/or the electrophysiology data displayed can be time varying or dynamic. Thus, in some embodiments, and as shown in FIG. 8, a time series of three-dimensional medical images 402a, 402b, 402c, and 402d, each with respective electrophysiology data displayed thereon, can be shown as a series of still frames 800a and/or as an animated image 402e in a separate window 800b, in a multi-pane or multi-window view as described above in connection with FIGS. 5 and 6. Likewise, it should be understood that the image slices 502, 504, 506 displayed can move as the preselected localization element moves.

Although several embodiments of this invention have been described above with a certain degree of particularity, those skilled in the art could make numerous alterations to the disclosed embodiments without departing from the spirit or scope of this invention.

For example, although certain embodiments described herein include only a single three-dimensional medical image, the teachings herein can be extended to multiple three-dimensional medical images, either simultaneously or on demand (e.g., the practitioner can toggle between two or more three-dimensional images).

All directional references (e.g., upper, lower, upward, downward, left, right, leftward, rightward, top, bottom, above, below, vertical, horizontal, clockwise, and counterclockwise) are only used for identification purposes to aid the reader's understanding of the present invention, and do not create limitations, particularly as to the position, orientation, or use of the invention. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other.

It is intended that all matter contained in the above description or shown in the accompanying drawings shall be interpreted as illustrative only and not limiting. Changes in detail or structure may be made without departing from the spirit of the invention as defined in the appended claims.

What is claimed is:

1. A method of displaying electrophysiology information, comprising:
registering a localization system to a three-dimensional medical image of an anatomical region;
localizing an electrophysiology catheter within the anatomical region using the localization system;
displaying a representation of the localization of the electrophysiology catheter on the three-dimensional medical image of the anatomical region; and
displaying one or more image slices of the three-dimensional medical image of the anatomical region, the one or more image slices being selected based upon the localization of the electrophysiology catheter.

2. The method according to claim 1, wherein the three-dimensional medical image of the anatomical region comprises a magnetic resonance image.

3. The method according to claim 1, wherein the three-dimensional medical image of the anatomical region comprises a computerized tomography image.

4. The method according to claim 1, wherein registering a localization system to the three-dimensional medical image of the anatomical region comprises using a non-rigid transformation to register the localization system to the three-dimensional medical image of the anatomical region.

5. The method according to claim 4, wherein the non-rigid transformation comprises one or more of a thin plate splines transform, a radial basis function transform, and a mean value coordinate transform.

6. The method according to claim 1, wherein registering a localization system to the three-dimensional medical image of the anatomical region comprises using a rigid transformation to register the localization system to the three-dimensional medical image of the anatomical region.

7. The method according to claim 1, wherein displaying one or more image slices of the three-dimensional medical image of the anatomical region, the one or more image slices being selected based upon the localization of the electrophysiology catheter, comprises displaying one or more image slices of the three-dimensional medical image of the anatomical region passing through a location of a preselected localization element carried by the electrophysiology catheter.

8. The method according to claim 1, further comprising displaying electrophysiology data measured by the electrophysiology catheter on the three-dimensional medical image of the anatomical region.

9. The method according to claim 1, further comprising displaying electrophysiology data measured by the electrophysiology catheter on the one or more image slices of the three-dimensional medical image of the anatomical region.

10. The method according to claim 9, wherein the electrophysiology data is displayed on the one or more image slices of the three-dimensional medical image of the anatomical region using a plurality of glyphs.

11. The method according to claim 1, further comprising displaying a scalar map on the three-dimensional medical image of the anatomical region.

12. The method according to claim 1, wherein obtaining a three-dimensional medical image of an anatomical region comprises obtaining a plurality of time-varying three-dimensional medical images of the anatomical region.

13. The method according to claim 1, further comprising displaying a graphical representation of the localization of the electrophysiology catheter on a model of the anatomical region generated by the localization system.

14. A method of displaying electrophysiology information, comprising:
registering a localization system to a three-dimensional medical image of an anatomical region;
localizing an electrophysiology catheter within the anatomical region using the localization system, the electrophysiology catheter including at least one localization element;
displaying one or more image slices of the three-dimensional medical image of the anatomical region, wherein the one or more image slices pass through a localization of the at least one localization element; and
displaying electrophysiology information measured by the electrophysiology catheter on at least one of the three-dimensional medical image of the anatomical region and the one or more image slices of the three-dimensional medical image of the anatomical region.

15. The method according to claim 14, further comprising displaying a representation of the localization of the electrophysiology catheter on at least one of the three-dimensional medical image of the anatomical region and the one or more image slices of the three-dimensional medical image of the anatomical region.

16. The method according to claim 14, wherein registering a localization system to the three-dimensional medical image of the anatomical region comprises registering the localization system to the three-dimensional medical image of the anatomical region using a non-rigid transformation.

17. The method according to claim 16, wherein the non-rigid transformation is selected from the group consisting of thin plate splines transforms, radial basis function transforms, and mean value coordinate transforms.

18. The method according to claim 14, wherein the three-dimensional medical image of the anatomical region is selected from the group consisting of computerized tomography images, magnetic resonance images, ultrasound images, x-ray images, fluoroscopic images, image templates, localization system generated images, segmented models of any of the foregoing, and any combinations thereof.

19. An electrophysiology system, comprising:
a localization system configured to localize an electrophysiology catheter;
a registration processor configured to register the localization system to a three-dimensional medical image of an anatomical region; and
a mapping processor configured to display:
a representation of a localization of the electrophysiology catheter on the three-dimensional medical image of the anatomical region; and
one or more image slices of the three-dimensional medical image of the anatomical region, the one or more image slices being selected based upon the localization of the electrophysiology catheter.

20. The system according to claim 19, wherein the mapping processor is further configured to display electrophysiology data measured by the electrophysiology catheter on at least one of the three-dimensional medical image of the anatomical region and the one or more image slices of the three-dimensional medical image of the anatomical region.

21. The system according to claim 19, wherein the representation of the localization of the electrophysiology catheter and the one or more image slices of the three-dimensional medical image of the anatomical region are time-varying.
